(19) United States
(12) Patent Application Publication
(10) Pub. No.: US 2002/0122835 A1
(43) Pub. Date: Sep. 5, 2002

(54) Compositions for enhancing muscle tissue recovery and repair

(76) Inventors: Luke R. Bucci, Reno, NV (US); Alan Shugarman, Salt Lake City, UT (US); Jeffrey A. Felliciano, West Hollywood, CA (US); Marta Draper Rodriguez, Salt Lake City, UT (US)

Correspondence Address: NIXON & VANDERHYE P.C., 1100 North Glebe Road, 8th Floor, Arlington, VA, 22201, US

(21) Appl. No.: 09/795,973

(22) Filed: Mar. 1, 2001

(51) Int. Cl. A61K 35/78; A61K 31/315; A61K 31/198

(52) U.S. Cl. 424/729; 424/733; 424/734; 424/741; 424/764; 514/494; 514/561

(57) ABSTRACT

Compositions comprising zinc, magnesium and vitamin B₆ and optionally theanine are effective for enhancing muscle and tissue recovery during sleep, following intense physical exercise.

The present invention relates to compositions for enhancing muscle and tissue recovery and repair. More particularly, the present invention provides compositions containing zinc monomethionine aspartate, magnesium, and vitamin B₆ which assist in muscle and tissue recovery particularly during sleep, following physical exercise.

### BACKGROUND OF THE INVENTION

Zinc and magnesium are important elements in body function. It is known for example that zinc plays a role in the level of circulating human Growth Hormone (hGH) in the body. Case studies have established that administration of zinc to patients deficient in hGH have shown a significant increase in growth rate as well as normalization of their hGH levels.

It is also known that a zinc has a positive influence on Insulin-like Growth Factor-I (IGF-1). IGF-I is an anabolic hormone for facilitating a growth environment. Case studies have established that IGF-I levels may significantly increase when subjects are supplemented with zinc.

Deficiencies in magnesium decrease oxygen delivery to muscle tissue. In addition, magnesium activates enzymes necessary for the metabolism of carbohydrates and amino acids.

A common observation amongst athletes through blood and urine testing is that they are deficient in zinc and magnesium. Zinc and magnesium losses are particularly noticeable following vigorous exercise and excessive sweating. Losses in zinc and magnesium may also lead to muscular fatigue and reduced endurance.

An approach which has been suggested to correct this deficiency is administration of zinc monomethionine aspartate along with magnesium aspartate and vitamin B₆. Case studies have established that athletes initially deficient in zinc and magnesium following administration of zinc monomethionine aspartate along with magnesium aspartate and vitamin B₆ have later exhibited normal zinc and magnesium levels.

Further studies have shown the close relationship between zinc and serum testosterone levels which translate to strength and power of athletes. While testosterone is an important substance with regard to growth and repair of muscle tissue, such growth and repair of muscle is also dependent on other hormones such as hGH and IGF-I. In addition, many of the enzymes that prevent the build-up of lactic acid (the “fatigue” acids), require zinc.

A need exists for a formula which can effectively increase zinc and magnesium levels, anabolic hormone levels as well as strength and power in people undergoing physical exercise, especially athletes undergoing intense training, and which promotes tissue and muscle recovery and repair. The present invention seeks to satisfy that need.

### SUMMARY OF THE INVENTION

In a first aspect, the present invention provides a composition comprising zinc, magnesium and vitamin B₆, and optionally further including theanine.

In a further aspect there is provided a composition suitable for enhancing muscle tissue recovery and repair following physical exercise, comprising zinc monomethionine, zinc aspartate, L-theanine, magnesium aspartate and vitamin B₆.

In a yet further aspect, there is provided a composition suitable for enhancing muscle tissue recovery and repair following physical exercise, comprising zinc monomethionine aspartate, L-theanine, magnesium aspartate and vitamin B₆.

In another aspect, there is provided a composition of the invention in unit dosage form.

In yet another aspect, there is provided a method of treating a patient to enhance muscle tissue recovery and repair following physical exercise, particularly during sleep, comprising administering an effective amount of the composition of the invention to the patient.

### DETAILED DESCRIPTION OF THE INVENTION

The composition of the invention comprises zinc, magnesium and vitamin B₆, with the optional further presence of theanine. The composition is particularly effective for night-time use to enhance muscle recovery and repair following physical exercise, to induce relaxation of the body prior to and during sleep. In particular, L-theanine is present to aid in reduction of stress and anxiety from activities including physical activity and daily work, allowing for relaxation prior to sleep and therefore deeper more restful sleep.

The zinc is typically present in the composition as zinc monomethionine and zinc aspartate, or as zinc monomethionine aspartate. Other forms of zinc could include oxide, sulfate, chelates, gluconate, creatinate, and others.

A typical unit dosage form of the invention contains about 5-50 milligams, for example 20 to 40 milligrams, zinc, more usually about 30 milligrams per unit dosage form.

Theanine, when present, is typically present as L-theanine. L-theanine is an amino acid which may be found in green tea leaf. L-theanine is known to control excitement caused by caffeine, and animal experiments using L-theanine have confirmed it to be safe. Recent studies have shown that oral administration of L-theanine has caused a feeling of relaxation amongst human volunteers. Kobayashi et al, Nippon Nogeikagaku Kaisha, Vol. 72, No. 2, 153-157 (1998).

In one aspect, it has been found, according to the present invention, that surprising properties arise when L-theanine is administered with zinc and magnesium in terms of muscle recovery and repair during sleep, following intense physical exercise during the day. The compositions of the invention provide significant unexpected tissue and muscle repair while supporting the relaxation and reducing muscle cramping potential over that observed using zinc monomethionine aspartate, magnesium aspartate and vitamin B₆ without L-theanine. A typical unit dosage form of the invention contains about 10 mg to 2 grams, for example 50 mg to 1 gram L-theanine, typically about 200 milligrams L-theanine.

Other materials which may be used in the compositions of the invention, either in place of L-theanine or in combination therewith, are kava kava, melatonin, GABA, valerian, skull cap, chamomile or green tea. L-theanine is preferred. The theanone and/or kava kava, melatonin, GABA, valerian, skull cap, chamomile or green tea may be present in amounts ranging from 10 mg to 2 grams, for example 50 mg to 1 gram, typically about 200 milligrams. These compounds are present due to their property of relaxing the body and/or inducing sleep.

Magnesium is typically present as a salt, such as magnesium aspartate. Other forms of magnesium that could be used include: oxide, sulfate, chelates, gluconate, creatinate, and others.

A typical unit dosage form of the invention contains about 50 milligrams to 1 gram, for example 350 to 500 milligrams, magnesium, more usually about 450 milligrams.

Vitamin B₆ is a cofactor in protein metabolism, and further assists in achieving muscle and tissue recovery during sleep. Vitamin B₆ is typically present in the composition of the invention as pyridoxine hydrochloride. Other forms of B₆ include, pyridoxal-5-phosphate, pyridoxamine, and others.

A typical unit dosage form of the invention contains about 5 to 30 milligrams, for example 9 to 12 milligrams, vitamin B₆, more usually about 10.5 milligrams.

The compositions are formulated to be administered orally, as unit dosage forms. A typical unit dosage form is a capsules. Other forms may be used such as tablets, powders, liquids, gums, gels, and bars.

A capsule typically comprises vitamin B₆ (as pyridoxine hydrochloride) 10.5 mg; magnesium (as magnesium aspartate) 450 mg; zinc (as zinc monomethionine and zinc aspartate) 30 mg; L-theanine 200 mg.

The composition of the invention is typically administered one time daily, preferably in the evening between dinner and bedtime. This administration regimen produces optimum muscle and tissue recovery during sleep.

In a further aspect, the invention provides a method of treating a patient to enhance muscle and tissue recovery, particularly during sleep, comprising administering an effective amount of the composition of the invention to the patient prior to sleep. The method is typically carried out by administering orally one unit dosage form of the composition, usually three capsules, between dinner and bed-time. It is preferred that some time elapse following completion of dinner before the first capsule is taken (0.5-3 hours). Typically, a period of at least half an hour should elapse following dinner before the capsules are taken. It is further recommended that the dose be consumed approximately 30 to 60 minutes prior to bedtime. If dinner was completed at 7:00 pm, it is recommended that the capsules be taken not prior to 7:30 pm, more usually at about 8:00 pm.

The dosage may vary depending on the size of the person. A woman or small man of 150 lbs or less may only need to consume ⅔ of the recommended dosage. For example, instead of consume three capsule they would consume only two capsules. Conversely, a larger man or woman of greater than 200 lbs may need to consume 4/3 the dosage. For example, instead of consuming three capsules they would consume four capsules.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A composition comprising zinc, magnesium and vitamin B₆.

2. A composition according to claim 1, wherein said zinc is present as zinc monomethionine.

3. A composition according to claim 1, wherein said zinc is present as zinc monomethionine and zinc aspartate.

4. A composition according to claim 1, wherein said zinc is present as zinc monomethionine aspartate.

5. A composition according to claim 1, wherein said magnesium is present as magnesium aspartate.

6. A composition according to claim 1, and further comprising theanine.

7. A composition according to claim 6, wherein said theanine is present as L-theanine.

8. A composition according to claim 1, further comprising a compound selected from the group consisting of kava kava, melatonin, GABA, valerian, skull cap, chamomile and green tea.

9. A composition according to claim 1, in unit dosage form.

10. A composition according to claim 9, wherein said zinc is present in an amount of about 5 to 50 milligrams.

11. A composition according to claim 10, wherein said zinc is present in an amount of about 30 milligrams.

12. A composition according to claim 9, wherein said theanine is present as L-theanine in an amount of about 10 mg to 2 grams.

13. A composition according to claim 12, wherein said L-theanine is present in an amount of about 200 milligrams.

14. A composition according to claim 9, wherein said magnesium is present in an amount of about 50 mg to 1 gram.

15. A composition according to claim 14, wherein said magnesium is present in an amount of about 450 milligrams.

16. A composition according to claim 9, wherein said vitamin B₆ is present in an amount of about 5 to 30 milligrams.

17. A composition according to claim 16, wherein said vitamin B₆ is present in an amount of about 10.5 milligrams.

18. A composition comprising zinc monomethionine, zinc aspartate, L-theanine, magnesium aspartate and vitamin B₆.

19. A composition comprising zinc monomethionine aspartate, L-theanine, magnesium aspartate and vitamin B₆.

20. A composition according to claim 9, comprising vitamin B₆ in an amount of about 10.5 mg, magnesium in an amount of about 450 mg, zinc in an amount of about 30 mg, and L-theanine in an amount of about 200 mg.

21. A method of treating a patient to enhance muscle and tissue recovery, comprising administering to said patient an effective amount of a composition according to claim 1.

22. A method according to claim 21, wherein a unit dosage form is administered orally between dinner and bed-time.

23. A method according to claim 22, wherein said unit dosage form is administered orally three times between dinner and bed-time.

24. A method according to claim 23 wherein said unit dosage form comprises vitamin B₆ in an amount of about 10.5 mg, magnesium in an amount of about 450 mg, zinc in an amount of about 30 mg, and L-theanine in an amount of about 200 mg.
